(19) United States
(12) Patent Application Publication
(10) Pub. No.: US 2009/0192648 A1
(43) Pub. Date: Jul. 30, 2009

(54) MEDICATION COMPLIANCE MANAGEMENT SYSTEM

(75) Inventors: Pavan K. Namineni, Ann Arbor, MI (US); James Burkowski, Northville, MI (US); Charles J. Jacobus, Ann Arbor, MI (US); Joe Long, Ypsilanti, MI (US); Gary Siebert, Dexter, MI (US); Trevor Davey, Canton, MI (US)

Correspondence Address: GIFFORD, KRASS, SPRINKLE,ANDERSON & CITKOWSKI, P.C, PO BOX 7021, TROY, MI, 48007-7021, US

(73) Assignee: Cybernet Systems Corporation, Ann Arbor, MI (US)

(21) Appl. No.: 12/026,117

(22) Filed: Feb. 5, 2008

Related U.S. Application Data
(60) Provisional application No. 60/888,156, filed on Feb. 5, 2007.

(51) Int. Cl. G06F 17/00; A61M 31/00

(52) U.S. Cl. 700/231; 604/48

(57) ABSTRACT

Methods and systems tell patients when to take their medications, control medication dispensing based on compliance to the prescribed dosage, and determine that the proper dosage has been taken. This system integrates with other devices related to home telehealth by including support for a world-wide web-based medical record associating the patient, his/her medication requirements or prescription, compliance with that prescription, and other associated medical telemetry data that may be used to determine patient response to said medication.

### REFERENCE TO RELATED APPLICATION

This application claims priority from U.S. Provisional Patent Application Ser. No. 60/888,156, filed Feb. 5, 2007, the entire content of which is incorporated herein by reference.

### FIELD OF THE INVENTION

This invention relates generally to medication compliance management and, more particularly, to methods and systems for cueing patients when to take their medications, controlling medication dispensing based on compliance to the prescribed dosage, and determining that the dosage has been take by the patient.

### BACKGROUND OF THE INVENTION

There are many devices designed to control medication dispensing. The field may be segmented into (a) devices that are stand alone and must be pre-programmed for reminders, etc. and are also examined by bring the device back to it point of origin to determine medication compliance, and (b) devices that have a network capability so that programming and compliance can be remotely monitored. Aten et al. U.S. Pat. No. 4,674,652 is an example of the first case and Rose et al. U.S. Pat. No. 4,695,954 is an example of the second case. Many newer disclosures (1998 and later) appear to incorporate some option for remote control or integration into some type of record keeping system.

Physically, such devices can be separated into (a) those that support preparation of medication in a pharmacy for distribution to the patient (pharmacy automation systems)—one such is described in Williams et al. U.S. Pat. No. 5,646,912 and Liff et al. U.S. Pat. No. 5,713,485; (b) those that are designed to support presentment of medications to patients in a hospital or medical care setting (and therefore support multiple medication to multiple patients, typically focusing on prevention of medication error making by the medical staff)—one such is described in Halvorson U.S. Pat. No. 4,847,764; (c) medication dispenser design for patient use in the home (or sometimes assisted living)—these device are for single patient users (many including U.S. Pat. Nos. 4,939,705, 5,752,620, 5,710,551, 5,751,661, etc.); and (d) devices that are design to provide medication mixes versus those design to provide and monitor compliance to a single medication (many including U.S. Pat. Nos. 4,674,652, 4,748,600, 4,911,327, 5,159,581. 5,246,136, 5,752,621, etc.).

The basic purpose of these devices is to (a) remind the patient to take medicine in compliance to prescription (first described in Carlson U.S. Pat. No. 4,223,801 and later in Aten et al. U.S. Pat. No. 4,674,652), (b) to assure that medicine can only be taken in a compliant manner (also described in Aten et al. U.S. Pat. No. 4,674,652), and (c) to determine that compliance has occurred (described in many cited patents, but in Aten et al. U.S. Pat. No. 4,674,652 as when the patient removes a medication vial). Some devices expect the patient to enter or notify his/her compliance (Hemmann, et al. U.S. Pat. No. 5,805,051), some measure compliance by (a) measuring a medication preparation cycle as a compliant action (Aten et al. U.S. Pat. No. 4,674,652), (b) measuring a medication removal as a compliance action (taking off a vial lid Walters U.S. Pat. No. 5,751,661; raising the lid of the dispenser Ridgeway U.S. Pat. No. 5,710,551), or (c) indirectly measuring compliance (for instance by counting medication cap removals as a compliance action—Pearson U.S. Pat. No. 5,752,620).

Finally, some devices (a) exploit existing standard medical packaging (for instance pill vials Abdulhay et al. U.S. Pat. No. 7,213,721 or Walters U.S. Pat. No. 5,751,661), (b) some require use specialized packaging (for instance blister packs that fit the dispenser Parkhurst, Canadian Patent CA 2145193, 1993, Parkhurst et al. U.S. Pat. No. 5,412,372, Benouali U.S. Pat. No. 6,973,371, and Fabricius et al. U.S. Pat. No. 7,170,823), and (c) some require re-packaging or feeding of the medications (for instance dumping from a standard vial into the dispensing device in some device specific manner Aten et al. U.S. Pat. No. 4,674,652, Hasey U.S. Pat. No. 6,766,219).

Most of the personal medication dispensers disclosed fall into one only a few alternative mechanical architectures: (a) caps that fasten onto standard medical vials (Jean-Pierre 2004, Hamilton et al. U.S. Pat. No. 4,939,705, Walters U.S. Pat. No. 5,751,661); (b) controlled drawer or filing cabinet forms (Halvorson U.S. Pat. No. 4,847,764, Williams U.S. Pat. No. 5,263,596, Blechl, et al. U.S. Pat. No. 5,377,864, Fedor, et al. U.S. Pat. No. 5,790,409, Higham, et al. U.S. Pat. No. 5,745,366); (c) two dimensional matrices (usually with pop-up pill areas covers—Schwartz 2004, Agans U.S. Pat. No. 5,159,581, Parkhurst et al. U.S. Pat. No. 5,412,372, Ridgeway U.S. Pat. No. 5,710,551, Gilmore U.S. Pat. No. 6,138,865, Lucas U.S. Pat. No. 6,221,010); (d) rotating carrousels (Urquhart U.S. Pat. No. 4,748,600, Urquhart et al. U.S. Pat. No. 4,971,221, Loidl U.S. Pat. No. 5,246,136, Kurtenbach U.S. Pat. No. 5,582,323, Chorosinsici et al. U.S. Pat. No. 5,945,651, Shusterman U.S. Pat. No. 6,304,797, Sahai et al. U.S. Pat. No. 5,971,594, Papp et al. U.S. Pat. No. 6,439,422, Papp U.S. Pat. No. 6,848,593, Conley U.S. Pat. No. 7,044,302); (e) moving conveyors (with pill reservoirs separated by some means like a vials or paddle wheels, for instance Aten et al. U.S. Pat. No. 4,674,652 or Hasey U.S. Pat. No. 6,766,219); and (f) drop down vials/tubes or compartment systems where the pills are release to gravity feed into the discharge cup (Aten et al. U.S. Pat. No. 4,674,652, Abdulhay et al. U.S. Pat. No. 7,213,721).

As is apparent from the above, however, that there is no device that is (a) highly portable, (b) uses standard medical pill vials, (c) can accommodate multiple pill types, (d) counts pill dispenses pill-by-pill (as opposed to cap openings), (e) only allow dispenses that are consistent with medical prescription to the patient, (f) incorporated into a (wireless) network interface that can update vial programming and can relay pill dispensing at near real time rates to a centrally located secure network (web-based) medical record, which also accepts and integrates medical data taken by other remote devices describing the patient's condition coordinated with medical compliance data for that patient.

Combining these features into the size and low cost necessary for a cap-type dispenser has been beyond the state-of-the-art for prior systems. Therefore prior units were either built to be full featured at the expense of device size or had important features removed to keep cost and size small enough to be a medication cap form factor.

### SUMMARY OF THE INVENTION

This invention resides in a medication compliance management system and method of using the same. The system includes a base station including a first communications interface, and a pill dispensing cap adapted to replace the cap of a standard pill vial. The pill dispensing cap includes a pill transfer mechanism operative to load a single pill from the vial and dispense that pill to a user, and an interface configured to communicate with the base station and inform the base station that the single pill has been dispensed. In the preferred embodiment, the base station further includes a second communications interface operative to provide compliance information to a remote location.

To conserve battery power, the pill transfer mechanism relies on gravity to load a single pill from the vial and dispense that pill to a user. The transfer mechanism further includes an interchangeable chamber to accommodate pills of different sizes.

The first communications interface is preferably a short-range wireless communications interface, enabling compliance information to be automatically transferred to the base station when the pill dispenser in proximate to the base station. The second communications interface preferably uses a plain old telephone service (POTS) interface, allowing the most number of users to communicate with healthcare personnel at one or more remote locations.

In the preferred embodiment, the base station includes a code reader to determine the type of pill or other medication contained in the vial, and an interface allowing at least a portion of this information to be loaded into the pill dispensing cap. The pill dispensing cap preferably includes an audible or visual alert to remind a user to take the medication contained in the vial. The compliance information typically includes a time stamp indicating the time that a pill was taken, and/or the number of pills taken over a given period of time.

### BRIEF DESCRIPTION OF THE DRAWINGS

FIG. 1 shows a process to deliver medications to the patient, cue and monitor compliance, and keep compliance records using remotely access network databases housed on servers;

FIG. 2 shows a summary of how compliance data is captured, stored, and securely made available to clinical personnel;

FIG. 3 shows a pill dispenser assembly;

FIG. 4 shows the pill dispenser electronics;

FIG. 5 shows the pill dispensing operation;

FIG. 6 shows the pill dispenser loading operation;

FIG. 7 shows the hub or base module form;

FIG. 8 shows the hub or base module electronics; and

FIG. 9 is a table showing the dimensions of child resistant vials.

### DETAILED DESCRIPTION OF THE INVENTION

There are several applications that need methods to monitor and manage medication compliance. These include assembling medications for supply or resale in the pharmacy setting, delivering and monitoring the delivery of medication to patients in a hospital or group care setting (i.e. supporting accurate delivery to multiple patient from a shared delivery source or card), and assuring medication compliance to the individual patient in a home of assisted living setting. This invention resides in a medication compliance management device (MCMD) and system focused primarily on the third application: assuring medication compliance to the individual patient in a home of assisted living setting and secondarily supporting accurate home-based clinical trials of new medications.

To support these applications, the disclosed MCMD has the following features:
1. Tamper proof medication compliance measurement.
2. Accurate Medication Dispensing, one pill at a time.
3. Protection from Air and Moisture.
4. Modularity, Portability and Minimization of Costs. This includes a simplified dispenser and a medication container that is able to be mailed and is disposable.
5. Mechanisms for timely intervention (for non-compliance and regimen changes).
6. Reduction of technology barriers for Patient (Simple to use).
7. Reduction of invasiveness of monitoring medication compliance for patient and supply chain, such as the pharmacist.
8. Real-time collection of compliance data.
9. Dispensing and monitoring of Different kinds and sizes of pills, (tablets and capsules), yet having a system that is portable.
10. Remote or wireless setting of medication prescription type, dosage and prescribed time of dosage.
11. Patient reminding.
12. Support for use of standard medical vials.

An important aspect of the system architecture is the modularity and portability that is achieved by the secure wireless communications between the base module and multiple Dispenser Caps. This is in contrast to several existing systems that form a large package for multiple medications and similar functionalities.

The main components of the system are:
1. A Pill Dispenser module that is a miniaturized electromechanical device that reminds, facilitates, monitors and records medication dispensing. The device accounts for every single pill. This cap will also dispense different kinds of pills. Hence it can be used to monitor multiple medications.
2. A Base Module that may be affixed to a wall near a telephone or a similar device. The base module also connects to the dispenser cap to collect medication compliance data in real time using low power wireless connections.
3. A Computer Server, where health professionals monitoring the patient have access to the patient's actions as recorded by the Dispenser Cap and Base modules. The base module communicates with the computer server using any available types of communication links, including Plain Old Telephone System (POTS) line, broadband or cellular link.

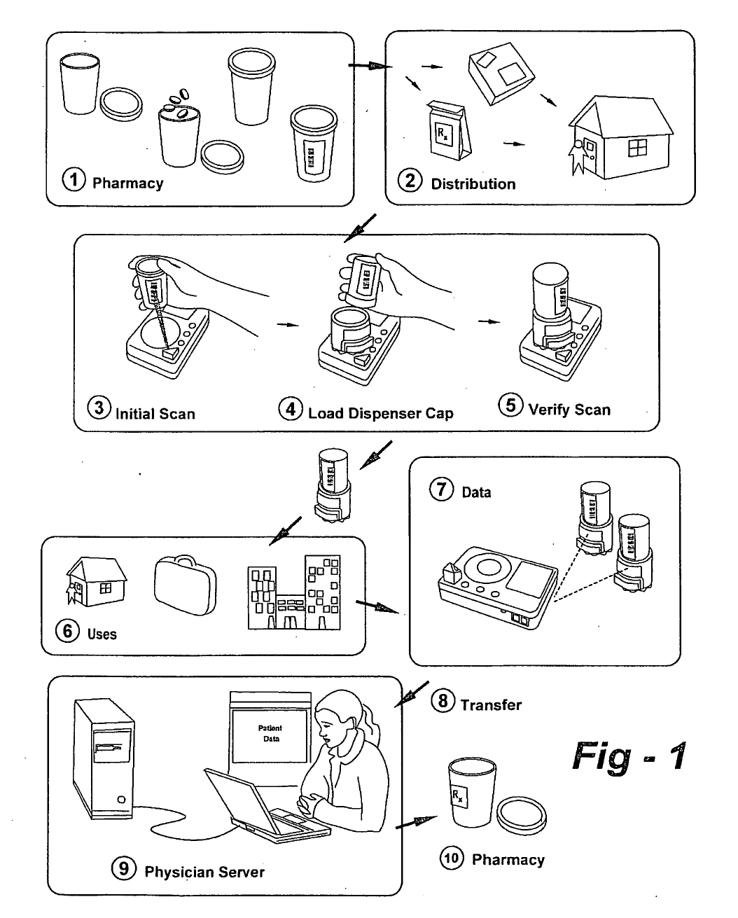

The disclosed medication cap/dispensing system is diagramed in FIG. 1. As shown in item (1), most pill pharmaceuticals are distributed in plastic cylindrical vials of several sizes, normally capped with snap-on or screw-on plastic tops. These are mailed to or picked-up by patients.

Either to assure medication compliance, or to automated the prescription refilling process, the invention incorporates a small and inexpensive automated medication vial cap that (a) fits the present standard vials, (b) supports multiple pill sizes and quantities, (c) can be programmed at home or at the pharmacy with the patient's prescription dosages and dose times, (d) provide patient reminders to take the medicine, (e) allows the patient to only take the prescribed dosages (within the prescribed dosage time window), (f) monitors that the dose are taken through direct pill counting at the time dispensing, (g) reports this data back to a central monitoring database (and clinical users that have access to this data) through a wireless network. The wireless network can also be used to insert medical dispenser cap programming and can assure that the proper physical vial is attached to the properly programmed cap.

Item (2) in FIG. 1 shows that the medical can be sent to the home user and he/she can attached the vial to the cap at home, using the label coded on the vial and scanned to set the cap programming as to dosage and dose timing. Alternatively, this can be done at the pharmacy and the entire cap-vial system can be shipped or otherwise delivered to the patient. The cost, battery endurance, and size goals for the cap automation support both of these approaches.

Items (3)-(5) in FIG. 1 shows how the bar coded label on the vial is verified at the time of cap/vial attachment through a scanner implemented into the medication hub or base module unit. After the vial is scanned, it is inserted into a cap that is properly programmed based on the vial scan (4), and vial and cap as a unit are verified (5) after affixment.

Item (6) in FIG. 1 shows that once the cap/vial assembly is affixed it can be used at home, during patient travel, or while at work just as any medical pill vial can be. However, when a pill is dispensed (described in greater detail below), the pill dispersal is time-stamped, counted, and entered into a non-volatile memory, which is part of (and internal to) the dispenser control microcomputer. When the pill dispenser is in near proximity to the patient's home-base medical base unit (7), it transmits its accumulated dispersal timestamps to the base unit. The base unit then (8) transmits this data through phone, broadband, cellular, or other communication means to a central data server.

The clinician or pharmacist can pull data records patient-by-patient and dispenser-by-dispenser (9) through a secure Internet connection (browser). This data can be (10) used for many useful medical applications, including (a) verifying that the patient is compliant with his/her medically prescribed treatment, (b) assuring that the patient receives timely refills, (c) providing an audit trail that a medical practitioner can correlate with other patient tests or physiological signs, and (d) providing statistical patient compliance data to minimize drug dosing errors.

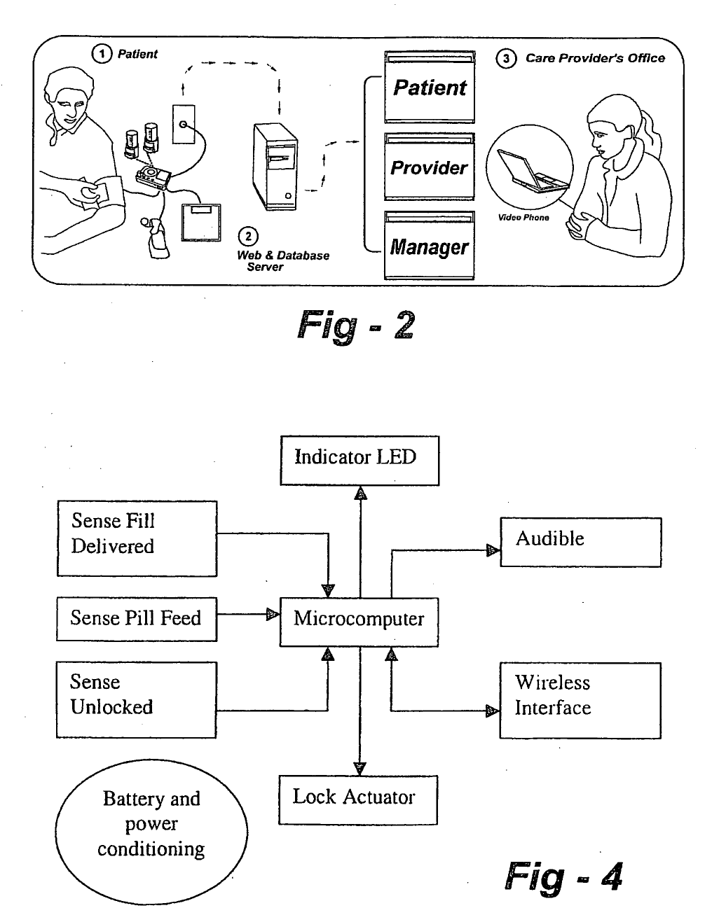

FIG. 2 shows the integration of this medical dispenser concept into a larger home-telehealth system architecture. A telehealth system will typically include a suite of medical measurement devices that a home patient can use to acquire aspects of their medical status (shown are blood pressure monitor and weight scale; others often include blood glucose, pulse oximetry, cardiac parameters or arrhythmias, etc.) and a means for effecting one-way or two-way communications through the communication network between the patient and his care giver—shown is a two-way video phone system, but this can also include specialized computer terminal devices, web pages, phone dialogs either between patient and computer or patient to clinical (i.e. a phone call center), as well as videophone.

In this system shown in FIG. 2 we add (1) the wireless medication dispenser that communicates into the system via the base module or hub. This adds one additional data stream to those already part of the over telehealth system. All devices, including the medication dispenser, communicate into the base module or hub (1), which then malces a common carrier communication connection (broadband, cellular, plain phone, or other) to the central data server (2). Then on-demand, clinical staff people can interrogate or in some other way utilize this data stream that now is augmented to include medication compliance information. Directly applicable to the medical dispenser is the typical telehealth system capability to screen incoming data so present the clinical staff with alerts, which for a medication dispenser would be associated with patient non-compliance with his or her prescribed medication treatment plan (i.e. what medicine to taken at what time and in what quantity).

The Web and Database Servers and Web Server Software is based on industry standards, which are FDA approved, HIPAA compliant, and are being used by many telehealth patients and clinical users.

The data collection server collects data from a plurality of base modules. The data collection server has an open interface to enable connectivity to legacy systems. It includes software for a Medical Database to include website specific interfaces for patients, Pharmacists, Practitioners/Nurses, and System Management (for inventory, billing, insurance, and user management). All user types (Providers, Patients, Managers) have secure password/login ID entrance portals.

Medical Staff Login Pages have Prescription Pages (for the medicine compliance and cap programming function), Procedure Pages, Patient Information Pages, Data Report Pages, Medication Compliance Report pages, Device Traclcing Pages, Report Generation, Compliance Reports, Manual Data Entry Pages and PDA/WAP support for Provider's pagers. Patient Login Pages include Patient Information Pages and Data Report Pages. Management & Provider staff has the ability to add/archive physicians, pharmacists, etc., enter prescription data (times, types, dosages, etc.), add new device types into the device management capabilities, perform minor cosmetic changes (screen and report layouts), and can perform Provider, Patient, and Medical Staff tasks with Manual Data Entry Pages, Compliance and Patient data reports, Global Inventory Management and Insurance Provider Management. The Database will include all fields requested, plus will include Transaction Log and History Tables.

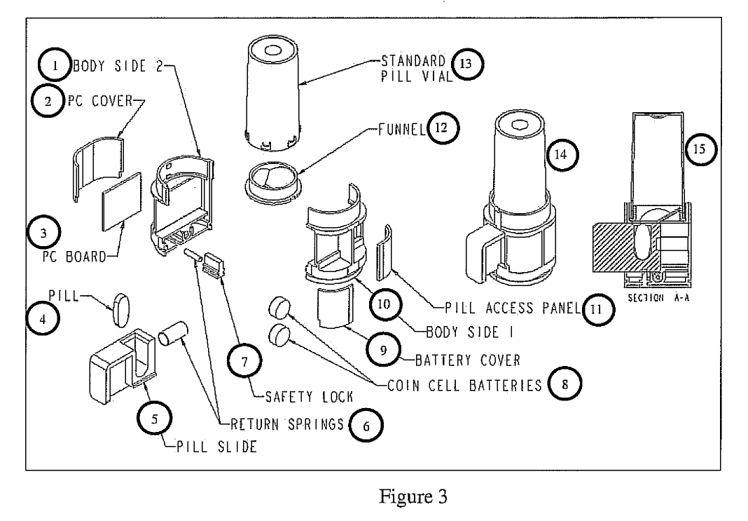

The Pill Dispenser is the device shown in FIG. 3 that replaces the cap of an existing standard pill vial. It is designed to separate a select pill type into a single order and allows only one pill to be accessed by a patient and electronically recorded. It is designed to be compact for traveling with the patient and can be stowed in any small handbag or coat pocket, the same as any stand-alone pill vial.

The Pill Dispenser twists onto a pill vial (13) that has been prescribed with the full prescription quantity before any pills have been administered by the patient. The patient removes the standard pill vial cap after receiving a prescription and replacing it with the Pill Dispenser or this operation being done by the pharmacist prior to deliver of the combination to the patient.

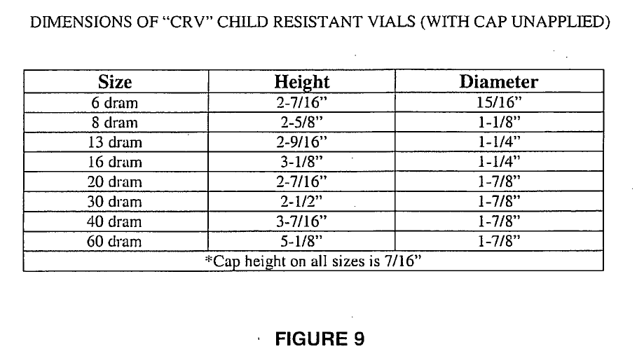

In this disclosure, “standard pill vial” is intended to mean existing as well as yet-to be-developed pill containers in which prescription and over-the-counter medications are provided. With appropriate dimensional modification that would be apparent to a person of sldll in the art, the Pill Dispenser is adaptable to non-child resistant, screw-on and child-resistant container varieties. By way of example, FIG. 9 is a table showing the dimensions of child resistant vials (CRVs) available from one source, namely Pharmacy Lite. Such vials currently feature caps with six inwardly protruding tabs and a compressible/resilient insert. The plastic vial itself has six outwardly oriented inverted U-shaped tab-receiving features and pointed ramps requiring the user to “push down and turn” the cap to get it off. Again, however, this is just one style of “standard” vial that will accommodate the Pill Dispenser.

The Pill Dispenser components are as follows: 2 Body halves (1) and (10), an Orientating Funnel (12), an Interchangeable Slider (5) with a Spring Return (6), a Slider Safety Lock (7) with Spring Return (6), a Dispensed Pill Access Panel (11), a Batteries (8) and a Cover (9), a PC-Board (3) and Cover (2) and screws to hold the assembly together. (4) shows a typical pill that fits the slider (5). (14) and (15) show the device assembled in perspective and as a side section, respectively.

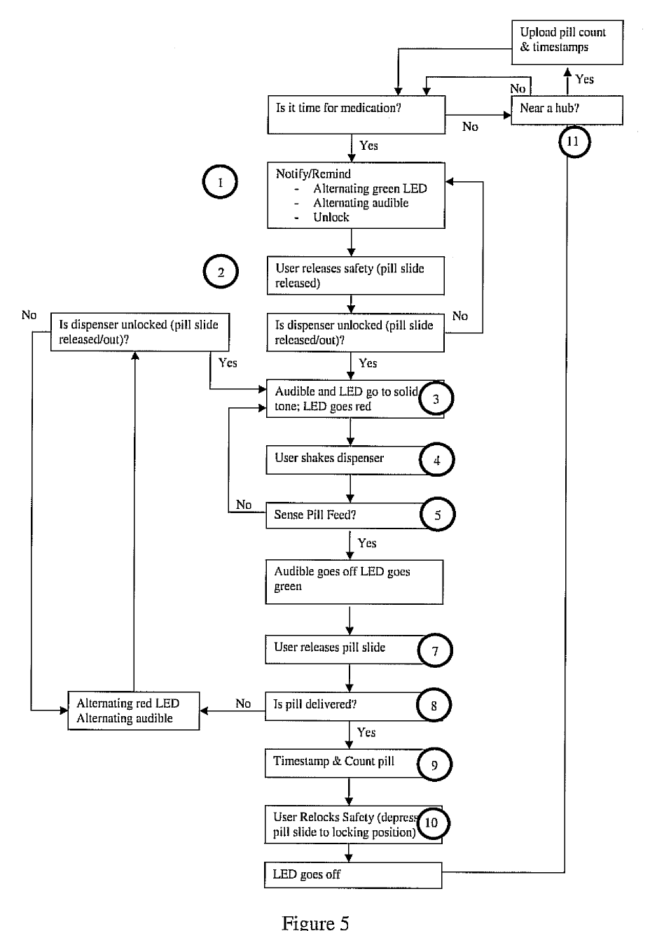

FIG. 4 shows the electronic system that is housed primarily on the PC board (FIG. 3 item (3)). The microcomputer includes I/O interfaces, internal non-volatile memory, and computation. The processor selected also includes a wireless interface and network software stack and interface so it can communicate with the base module. The microcomputer can lock the pill slider mechanism (lock actuator) until it is time to allow the patient to feed a dosage. The system can use both the audible signaling device and a Red/Green Indicator LED to communicate dispenser status to the patient of the pharmacist that program the prescription into the device. It has small optical presence/absence sensor to indicate that a pill has fed from the pill vial to the slider (Sense Pill Feed), that the pill slide is unlocked (Sense Unlocked), and that a pill is dispensed through the pill access panel to the patient (Sense Pill Delivered).

FIG. 5 describes how the patient operates the Pill Dispenser. Typically the dispenser indicates through audible and LED indication that it is time to take a medication dose (1). The patient releases the pill slide by first pushing the Safety Lock button located at the bottom of the unit (2). The Safety Lock is spring-loaded and requires the patient to push it to the open position, which allows the Pill Slide to automatically slide open from its closed position. The Pill Slide is typically stowed in the closed position when not in use as a matter to keep the device more compact. The Safety Lock will automatically return to the closed position when let go by the patient and the Pill Slide will be in the open position.

When the Safety is un-Locked, the LED and audible indications change until a pill is properly dispensed. This allows the cavity of the Pill Slide to be exposed in order for the patient to lightly shake the device (4), causing a single Pill to Funnel downward into the cavity. The dimensions of the cavity are designed to the particular Pill prescribed to the patient. The boundary conditions of the Pill Slide cavity dimensions allow a single pill to be captured, separating it from the Pill Vial. When the PC-Board electronics detect that a Pill has been properly accepted into the cavity (5), a signal to the patient will be lighted (6). This will notify to the patient to proceed with dispensing the pill.

Pushing the external button of the Pill Slide linearly inward until the pill reaches the Pill Access area dispenses the Pill (7). The smooth top feature of the Pill Slide keeps the remaining Pills left in the vial. The separated Pill will move and be exposed behind the transparent plastic Pill Access Panel. The PC-Board electronics will also signal with an indication that the Pill is accessible and the patient will be able to open the Pill Access Panel to retrieve the prescribed Pill for ingestion (8). The PC-Board electronics will then tale account of the Pill being dispensed, as it is removed form the access compartment (9).

The Safety Lock then automatically reengages the locking feature and locks the Pill Slide in the closed position until the process needs to be repeated by the patient at the prescribed intervals (10). As part of return to its original operational state, the dispenser interrogates it internal medicine dispensing event table, and if any data has been stored (i.e. a dispensing event had occurred), the PC board microcomputer imitates a query cycle to see if it is sufficiently near to a base module to upload the accumulated medicine dispensing event table (11). If so, this is done, otherwise the device returns to its quiescent sleep state to conserve battery power.

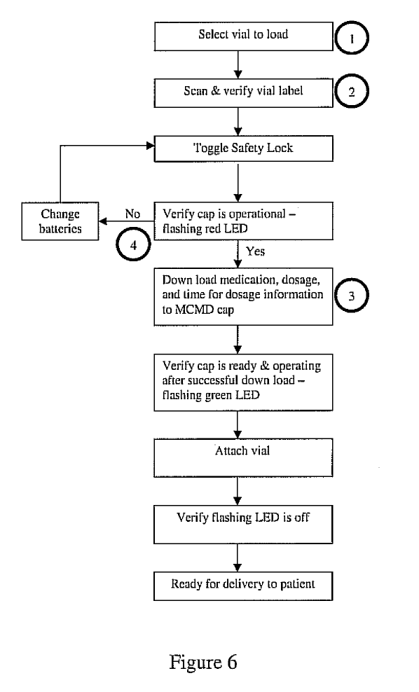

FIG. 6 summarizes how the medicine dispenser is filled with a new prescription. The prescription consists of a medical vial (1) containing a certain number of pills. Typically, the number of doses, the medication type/name, and its dosage timing are indicated on the vial label. This data will have also been entered into the centralized on-line database associated with the patient and the medication vial. Thus, the label data can be scanned (2) using a barcode or equivalent means, the barcode data can be used to query the centralized online data base, and from the retrieved data, the medicine dispenser programming parameters can be determined and down loaded into the medication dispense (3). As part of this operation, FIG. 6 (4), the pharmacist might have to change vial batteries (FIG. 3 (8)) and/or the version of the pill slide (FIG. 3 (5)) that fills the pill shape that is in the prescription.

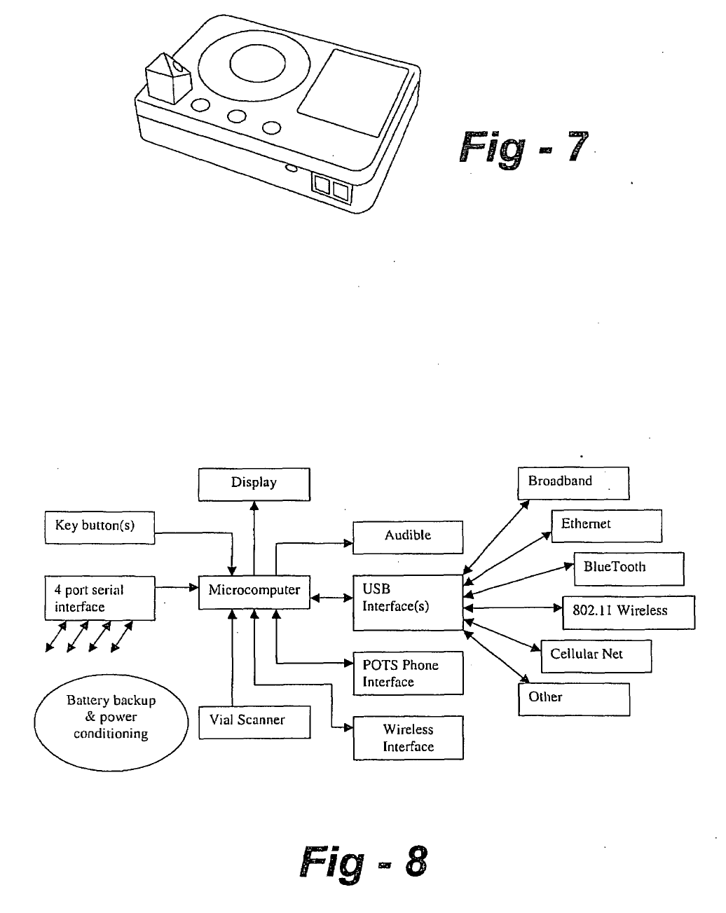

Referring to FIG. 7, the Base Module has a user display, audible alert(s), buttons to interact with the user, a scanner for reading the pill vial label (optional), and serial ports to read other medical devices (optional), uplink ports (USB, broadband, plain phone, or other), and a wireless link interface to read/write data to proximal medical dispenser caps.

Referring to FIG. 8, the microcomputer-based base module electronics is used to collect the compliance data from multiple dispenser caps using low power, short range wireless communications (Wireless Interface) and transmit the data via a Broadband, Cellular, BlueTooth, 802.11 Wireless, Plain Old Telephone Service (POTS Phone Interface) to a Data Collection Server listening on a toll-free number, or other communications means. It has a user display, audible, and key buttons to interact with the user. The base module includes a mechanism/method such as a bar code scanner (Vial Scanner) to identify the medication in the pill bottle. It coordinates with the Pill Dispenser Caps to guide the user to intuitively use the right bottle with the right dispenser cap. It is designed to be an automatically foolproof system. For flexibility, many of the communications options are implemented through a USB interface so that commercial interfaces built for PCs can be used. To enhance reliability the unit includes a battery back-up energy source so that it can work through intermittent power or portable applications.

The base module uplinks (coordinates) with the web servers through the data collection servers for messages, alerts and changes in medication regimen. The base module uses its LCD screen to display messages (1.) To guide the user and (2.) to provide any tailored messaging or counseling. The base module has an audible for interaction. In the optional form the base unit may also include additional (serial) ports or wireless protocols for acquiring and relaying data from other medical devices to the central servers used in a telehealth applications. The barcode scanning sensor can be removed if human operator (patient or pharmacist) checking/validation is substituted. Or the code can be in another form using alternative means of sensing, such as RFID (Radio Frequency Identification Devices).

We claim:

1. A medication compliance management system, comprising:
a base station including a first communications interface; and
a pill dispensing cap adapted to replace the cap of a standard pill vial, the pill dispensing cap including:
a pill transfer mechanism operative to load a single pill from the vial and dispense that pill to a user, and
an interface configured to communicate with the base station and inform the base station that the single pill has been dispensed.

2. The medication compliance management system of claim 1, wherein the base station further including a second communications interface operative to provide compliance information to a remote location.

3. The medication compliance management system of claim 1, wherein the pill transfer mechanism relies on gravity to load a single pill from the vial and dispense that pill to a user.

4. The medication compliance management system of claim 1, wherein the pill transfer mechanism includes an interchangeable chamber to accommodate pills of different sizes.

5. The medication compliance management system of claim 1, wherein the first communications interface is a short-range wireless communications interface.

6. The medication compliance management system of claim 1, wherein:
the first communications interface is a short-range wireless communications interface; and
compliance information is automatically transferred to the base station when the pill dispenser in proximate to the base station.

7. The medication compliance management system of claim 2, wherein the second communications interface uses a plain old telephone service (POTS) interface.

8. The medication compliance management system of claim 1, wherein the base station includes a code reader to determine the type of pill or other medication contained in the vial.

9. The medication compliance management system of claim 1, wherein:
the base station includes a code reader to determine the type of pill or other medication contained in the vial; and
the base station loads at least a portion on this information into the pill dispensing cap.

10. The medication compliance management system of claim 1, wherein the pill dispensing cap includes an audible or visual alert to remind a user to tale the medication contained in the vial.

11. The medication compliance management system of claim 1, wherein the compliance information includes a time stamp indicating the time that a pill was taken.

12. The medication compliance management system of claim 1, wherein the compliance information includes the number of pills taken over a given period of time.
